(19) United States
(12) Patent Application Publication
(10) Pub. No.: US 2025/0325398 A1
(43) Pub. Date: Oct. 23, 2025

(54) SYSTEM FOR TREATMENT OF PERIPHERAL NEUROPATHY

(71) Applicant: Auburn University, Auburn, AL (US)

(72) Inventors: Jonathan C. Commander, Auburn, AL (US); Chad Rose, Auburn, AL (US); Rhet Hailey, Auburn, AL (US); Michael E. Zabala, Auburn, AL (US); Vitale K. Castellano, Auburn, AL (US); Thomas E. Burch, Auburn, AL (US)

(73) Assignee: Auburn University, Auburn, AL (US)

(21) Appl. No.: 19/180,660

(22) Filed: Apr. 16, 2025

Related U.S. Application Data
(60) Provisional application No. 63/637,580, filed on Apr. 23, 2024.

(51) Int. Cl. A61F 7/00; A61H 9/00; A61H 23/02

(52) U.S. Cl. CPC A61F 7/007; A61H 9/0092; A61H 23/0254; A61F 2007/0071; A61H 2201/0207; A61H 2201/0228; A61H 2201/0242; A61H 2201/164; A61H 2201/165; A61H 2201/5082

(57) ABSTRACT

The treatment system includes a limb sleeve containing a heating subsystem, a pressure subsystem, and a vibration subsystem. The heating subsystem provides controlled cyclic heating to the limb, leading to dilation of the microvasculature and improved blood flow to nerve receptors. The pressure subsystem provides progressively staged, cyclic pressure to the limb, thereby improving blood circulation to the limb, reducing edema buildup, and flushing waste out the lymphatic system. The vibration subsystem provides controlled vibrotactile sensation to the limb to promote neuronal stimulation and decrease pain sensation. The treatment system allows for a multi-faceted approach in disease stabilization and assistive, personalized treatment for peripheral neuropathy.

### CROSS-REFERENCE TO RELATED APPLICATIONS

This application claims the benefit of prior-filed, co-pending U.S. Provisional Patent Application No. 63/637,580, filed on Apr. 23, 2024, the contents of which are incorporated herein by reference in their entirety.

### STATEMENT REGARDING FEDERALLY SPONSORED RESEARCH OR DEVELOPMENT

This invention was made with government support under TR003096 awarded by the National Institutes of Health. The government has certain rights in the invention.

### BACKGROUND OF THE INVENTION

The present application is directed to the field of non-invasive medical treatment. More specifically, the present application is directed to the field of non-invasive medical treatment of peripheral neuropathy.

Peripheral neuropathy (PN) is a common consequence of diabetes mellitus (DM) along with other ailments, most present in the older population with DM. Common symptoms of PN include numbness and loss of sensation, painful prickly sensations, burning, and pain. Varied combinations of these symptoms lead to a feedback loop with painful symptomatic repercussions discouraging an active lifestyle and allowing for reduction in vascular flow to the feet and increased peripheral edema. Degradation of gait and mobility leads to further increased exacerbation of edema and reduction in vascular flow resulting in poor balance, more falls, and increased risk of foot ulcerations.

Nerve deterioration in the periphery can be a direct result of lack of blood flow to the macro and microvasculature, which results in a reduction of nutrients to the nerves and accumulation of toxic elements. Structurally, PN results from an altered state to the vasculature, delayed blood transit, and ischemic condition in nerve fibers. More specifically, tissue hypoxia in neuronal and Schwann cells may exacerbate oxidative stress or capillary flow which results in increased heterogeneity and disproportionate nutrient transfer. Increased blood flow to the vasculature will increase nutrients. Along with a lack of blood flow from the disrupted capillary flow, edema accumulation in the peripheral limbs leads to less dilation of the arteries and reduces venous blood flow from the feet further compromising the feet. This impairment increases demyelination and buildup of neurotoxins, and reduces nerve conduction. Nerve conduction velocity follows altered or impaired vasodilation and decreases flow to the epineurial arteries with microvascular changes occurring early in the DM life-cycle.

Affecting around 50% of diabetic adults during their lifetime, PN leads to large nerve ending disease, causing pains that may lead to foot ulcers and limb loss associated with PN. Current treatment methods rely heavily on pharmaceutical pain management methods which include anti-depressants, anti-inflammatories, anti-convulsants, and opiates. Therapy and prescription drugs may treat symptoms, but the use of pharmacological medicine to treat symptoms does not treat the underlying damage to the neural pathways leading to the lower-limbs. Typically, PN symptoms are of the primary focus as a pain management, since PN can lead into such a painful, debilitating cycle. Pain in the peripheral limbs is typically shown in 30% of PN patients and treated with pharmaceuticals such as tricyclic antidepressants, anticonvulsants, and serotonic-norepinephrine reuptake inhibitors. These current pain treatment methods invoke multiple side effects such as loss of balance, decrease in mentation, sedation, and addiction. Decreased mentation can cause poor judgment and confusion about taking the drugs. It may also lead to increased fall risk, worsening personal health care, increased frequency of doctors' visits, inability to seek help, and poor hygiene, especially of the feet.

Alternative therapy methods for PN treatment have been proposed, including meditation, whole-body-vibration, neurostimulation, and increased vitamin-D intake. Typically relying on anecdotal feedback, these methods lack extensive support in the literature to show statistical improvement, and usually are paired with a healthy lifestyle for pain healing remedies. These methods are not linked to specific nerve healing or treatment and exist as alternative, non-traditional methods.

It is therefore the object of this application to provide a system for non-invasive medical treatment of peripheral neuropathy.

### BRIEF SUMMARY OF THE INVENTION

A system for treatment of peripheral neuropathy includes a heating subsystem having at least one heating element, a pressure subsystem having a plurality of pressure chambers, a vibration subsystem having a plurality of vibrational actuators, a limb sleeve containing the heating, pressure, and vibration subsystems, at least one controller operably connected to the heating, pressure, and vibration subsystems, and at least one power source.

The objects and advantages will appear more fully from the following detailed description made in conjunction with the accompanying drawings.

### BRIEF DESCRIPTION OF THE SEVERAL VIEWS OF THE DRAWINGS

FIG. 1a illustrates a schematic partially cross-sectional view of a system for treatment of peripheral neuropathy according to certain embodiments.

FIGS. 1b, 1c, and 1d illustrate schematic partially cross-sectional views of a heating subsystem, a pressure subsystem, and a vibration subsystem, respectively, according to certain embodiments.

FIG. 1e illustrates a schematic view of a portion of the vibration subsystem according to certain embodiments.

FIG. 2a illustrates an exemplary graph of the heating temperature in an embodiment of the heating subsystem as a function of time.

FIG. 2b illustrates an exemplary graph of the pressure exerted by each of the pressure chambers in an embodiment of the pressure subsystem as a function of time.

It should be understood that, for clarity, not all elements are labeled in all drawings. Lack of labeling in a figure should not be interpreted as lack of a feature.

### DETAILED DESCRIPTION OF THE INVENTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and/or methods described herein may be used alone or in combination with other systems and/or methods. Dimensions and materials identified in the drawings and applications are by way of example only and are not intended to limit the scope of the claimed invention. Any other dimensions and materials not consistent with the purpose of the present application can also be used. Various equivalents, alternatives and modifications are possible within the scope of the appended claims. Each limitation in the appended claims is intended to invoke interpretation under 35 U.S.C. § 112, sixth paragraph, only if the terms “means for” or “step for” are explicitly recited in the respective limitation.

Although there are no current wearable multi-treatment mode garments to the peripheral limbs, PN may potentially be treated via targeted stimulation to the nerve endings through an easy to wear, rehabilitative therapy treatment system 100. Through multiple targeted stimuli to encourage an increase in blood flow and reduction in edema buildup, the treatment system 100 is a systemic application device that provides a multi-faceted approach in disease stabilization and assistive, personalized treatment for type II DM individuals and others afflicted with PN. Furthermore, because microvascular disease can be present years before organ disease is recognized, use of the treatment system 100 might help preclude some end-stage organ disease, preventing consequences more widespread than harm to a patient's feet.

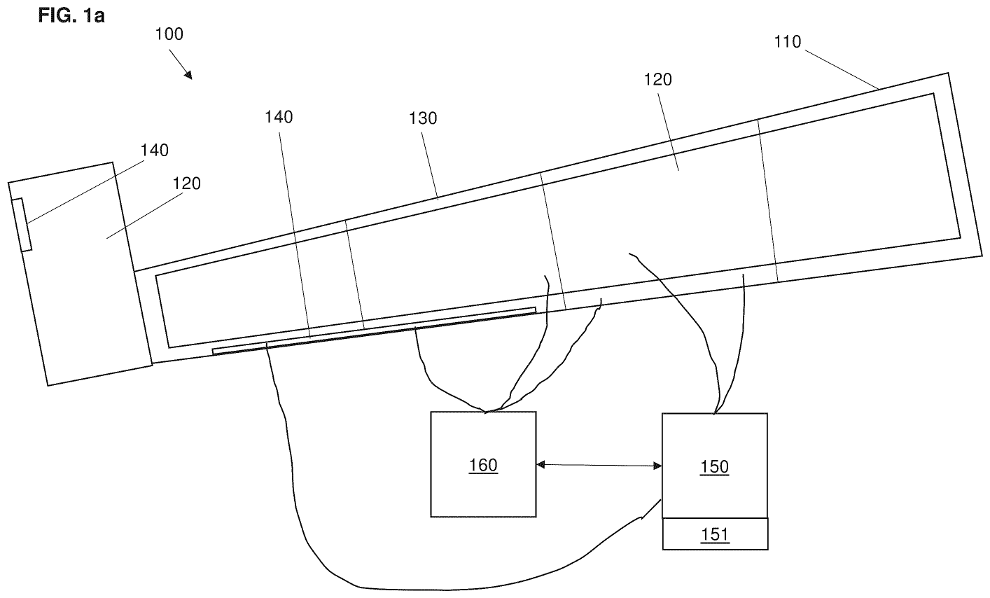

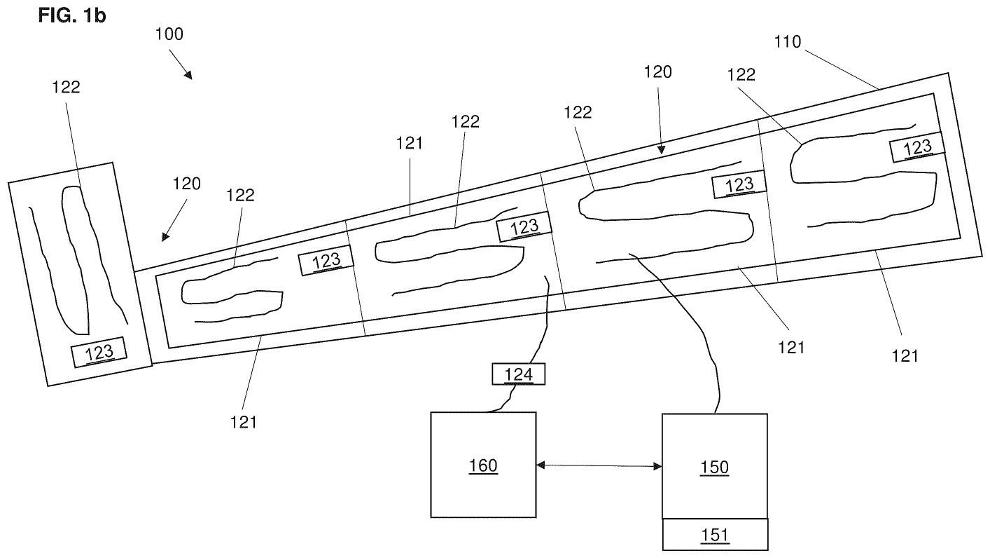

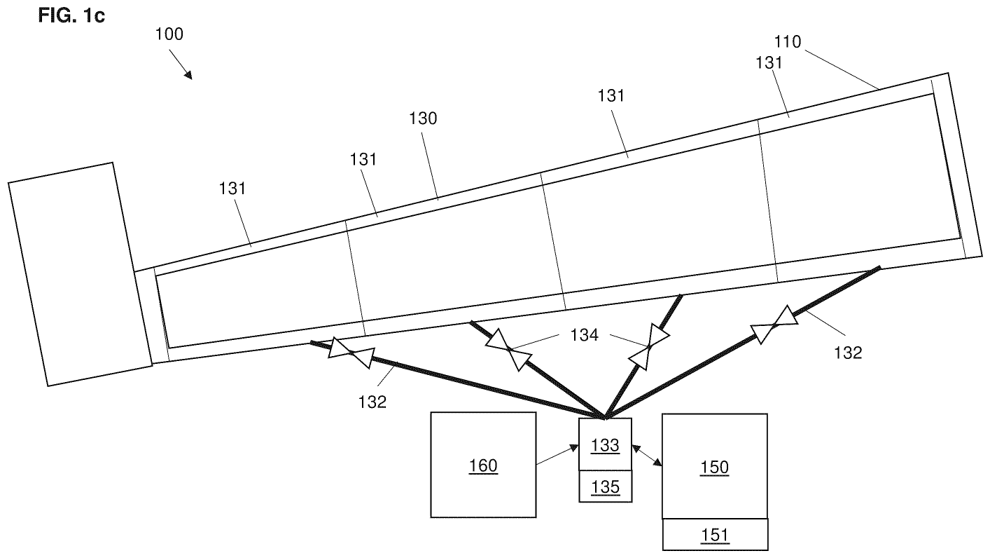

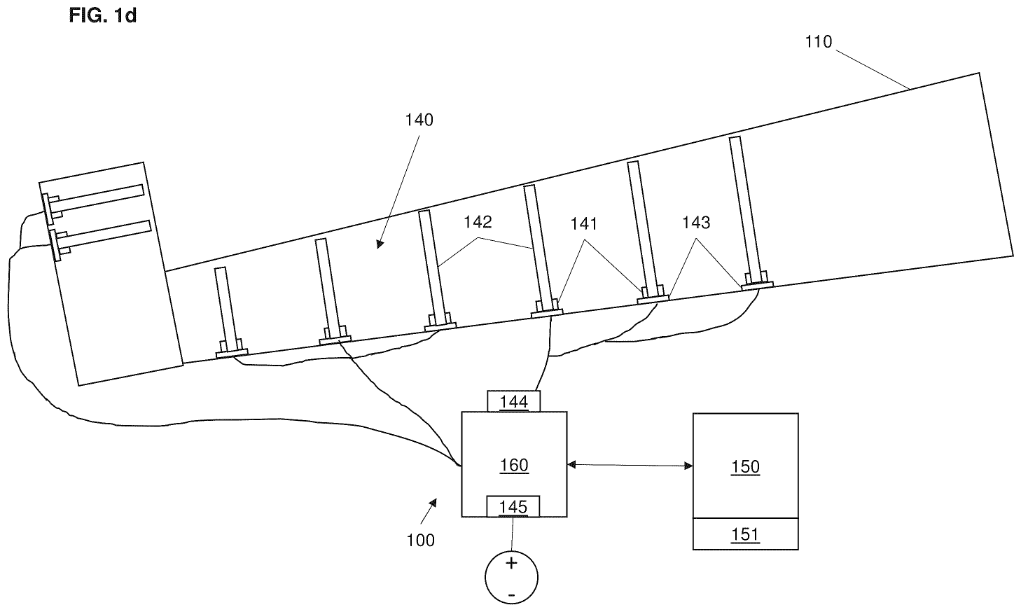

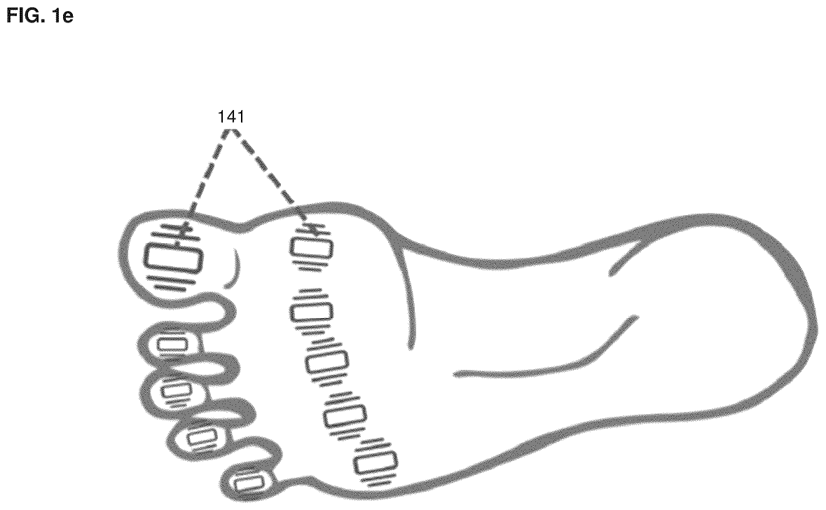

FIG. 1a illustrates a schematic view of a treatment system 100 for treatment of peripheral neuropathy according to certain embodiments. FIGS. 1b, 1c, and 1d illustrate schematic views of a heating subsystem 120, a pressure subsystem 130, and a vibration subsystem 140, respectively, according to certain embodiments. FIG. 1e illustrates a schematic view of a portion of the vibration subsystem 140 corresponding to the plantar surface of a patient's foot according to certain embodiments.

As shown in FIG. 1a, the treatment system 100 comprises a limb sleeve 110 containing the heating subsystem 120, the pressure subsystem 130, and the vibration subsystem 140. The heating subsystem 120, pressure subsystem 130, and vibration subsystem 140 are all operably connected to at least one controller 150 and at least one power source 160, both or either of which may be integrated with or separate from the limb sleeve 110. In certain embodiments, the controller 150 includes a user interface 151 capable of receiving user input and/or displaying system output.

The heating subsystem 120 provides controlled cyclic heating to the limb. Heat cycling is known to lead to dilation of the microvasculature and cause improved blood flow to nerve receptors, impacting sensation in the limb, such as on the plantar surface of the foot. The pressure subsystem 130 provides progressively staged, cyclic pressure to the limb. Staged cyclic compression, especially in combination with cyclic heating, improves blood circulation to the limb, reduces edema buildup, and flushes waste out the lymphatic system. The vibration subsystem 140 provides controlled vibrotactile sensation to the limb. Vibrotactics have been shown to promote neuronal stimulation and decrease pain sensation. It should be understood that while embodiments may refer to application of the treatment system 100 on a leg, alternate versions of the treatment system 100 may instead be applied to the arm. It should also be understood that reference to a limb may also include at least one appendage at the distal end of the limb.

The limb sleeve 110 is a removable sleeve extending at least partially around and at least partially along a limb. In the embodiment shown in FIG. 1a, the limb sleeve 110 extends from mid-thigh to the distal end of the foot, covering the phalanges. The limb sleeve 110 is at least partially constructed from a non-elastic or substantially non-elastic material.

As shown in FIG. 1b, the heating subsystem 120 includes at least one heating element 122 located between an outer surface of the limb and an inner surface of the limb sleeve 110. In certain embodiments, the heating element 122 is an electrically resistive heating element receiving power from the power source 160. In certain embodiments, the heating element 122 is a channel for the passage and circulation of a heated fluid, such as, but not limited to, heated water or gas. In certain embodiments, the heating element 122 is contained within at least one heating pad 121, which may be made from fabric or another material.

The heating subsystem 120 also includes at least one temperature sensor 123 located proximally to both the heating element 122 and the patient's skin surface. The temperature sensor 123 works in concert with the controller 150 to ensure that the temperature of the heating element 122 does not rise above a preselected maximum or fall below a preselected minimum. In embodiments with multiple heating elements 122, each heating element 122 is located proximally to its own temperature sensor 123. In an embodiment, the temperature sensor 123 is selected from thermistor-type sensors, thermocouples, resistance temperature detectors (RTDs), and other semiconductor-based integrated circuits (ICs). In certain embodiments, the heating subsystem 120 includes at least one temperature safety 124, an electrical or physical switch or interrupt, such as, but not limited to, a fuse or solid-state relay switch. The temperature safety 124 turns off all power to the heating subsystem 120 if the temperature of any heating element 122 detected by any temperature sensor 123 rises above a preselected emergency maximum. Such an event may trigger system output from the user interface 151.

As shown in FIG. 1c, the pressure subsystem 130 includes a plurality of pressure chambers 131 substantially encircling and sequentially extending along the limb, located between an outer surface of the limb and an inner surface of the limb sleeve 110. The pressure chambers 131 are hollow bladders receiving an inflation fluid, such as, but not limited to, water or gas. Since the limb sleeve 110 is at least partially constructed from an at least substantially non-elastic material, inflation of the pressure chambers 131 exerts a constrictive, “squeezing” pressure on the limb.

At least one length of pressure tubing 132 extends between the pressure chambers 131 and a pressure source 133, forming a conduit for inflation fluid from the pressure source 133 to the pressure chambers 131. The pressure source 133 may include a means for heating and/or cooling the inflation fluid. At least one actuatable pressure valve 134 in line with the pressure tubing 132 prevents premature deflation of the pressure chamber 131 and permits controllable deflation. In certain embodiments, each pressure chamber 131 is capable of inflation and deflation separate from any other pressure chamber 131. In certain embodiments, each pressure chamber 131 is operably connected to a pressure sensor 135 located in the pressure subsystem 130 and capable of monitoring pressure in the pressure chamber 131. In various embodiments, the pressure sensor 135 may be a strain gauge, piezoelectric sensor, micro-electromechanical system (MEMS) sensor, optical sensor, or capacitive sensor.

As shown in FIGS. 1d and 1e, in an embodiment, the vibration subsystem 140 includes a plurality of vibrational actuators 141 attached to motor straps 142 extending around the limb. Depending on the deployment pattern of the vibrational actuators 141, multiple vibrational actuators 141 or a single vibrational actuator 141 may be placed on a single motor strap 142. In an embodiment shown in FIG. 1e, multiple vibrational actuators 141 are placed on a single motor strap 142 on the patient's foot, spaced such that one vibrational actuator 141 is placed beneath each metatarsal head of the patient's foot. In an embodiment shown in FIG. 1e, multiple vibrational actuators 141 are placed on a single motor strap 142 on the patient's foot, spaced such that one vibrational actuator 141 is placed beneath each distal phalanx of the patient's foot. In an embodiment shown in FIG. 1d, multiple vibrational actuators 141 are placed on multiple motor straps 142 on the patient's calf, one vibrational actuator 141 on each motor strap 142. In such an embodiment, the motor straps 142 are spaced along the longitudinal axis of the limb, with the vibrational actuators 141 extending in a line along the patient's sural nerve. In an embodiment, the motor straps 142 are spaced equidistantly along the limb.

In an embodiment, the vibrational actuators 141 are commercially available vibromotors. In an embodiment, the vibrational actuators 141 are unbalanced oscillating motors. In an embodiment, the vibrational actuators 141 oscillate at a frequency of approximately 140 Hz. In an embodiment, the vibrational actuators 141 are actuated in a binary cycle fashion. In an embodiment, the vibrational actuators 141 are actuated on for a half second and off for a half second to ensure the maximum vibrational magnitude. The motor straps 142 are adjustable bands capable of encircling the patient's limb. In various embodiments, the motor straps 142 may be attached to, removable from, or separate from the limb sleeve 110. In various embodiments, the motor straps 142 may be elastic or inelastic material.

In certain embodiments, the vibrational actuators 141 are held in place on the strap by at least one motor holder 143. The motor holder 143 may be a molded or printed polymer member, and may have at least one connection feature for receiving or otherwise connecting to at least part of the vibrational actuator 141 and at least one slot through which the motor strap 142 slidably extends.

In an embodiment, each vibrational actuator 141 is controlled at a maximum actuator voltage of 3.3 volts and maximum actuator amperage 0.2 amps. In an embodiment, each vibrational actuator 141 is connected to the power source 160 by a vibrational actuator fuse 144 that does not allow for more than a maximum actuator amperage. In an embodiment, the power source 160 is stepped down from the line voltage to the maximum actuator voltage. In an embodiment, the power source 160 includes a main vibrational actuator fuse 145 that does not allow for more than a maximum total amperage distributed over all of the vibrational actuators 141. In an embodiment, the maximum total amperage is 5 amps.

In certain embodiments where the heating element 122 is a channel for the passage and circulation of a heated fluid, the heating subsystem 120 may be combined with the pressure subsystem 130 such that the heating elements 122 and pressure chambers 131 are the same component. In such an embodiment, pressure source 133 may supply the heated fluid or another pressure source 133 may supply the heated fluid.

In certain embodiments, the system 100 does not include motor straps 142 or motor holders 143. In these embodiments, the vibrational actuators 141 are fluid transport channels through which pulsatile fluid flow or gas bubbles may create controlled vibrotactile sensation to the limb. In such an embodiment, the pressure source 133 may supply the vibrational fluid or another pressure source 133 may supply the vibrational fluid.

As shown in FIG. 1a, the controller 150 is operably connected to the heating subsystem 120, pressure subsystem 130, and vibration subsystem 140 to control operation of all three subsystems. In certain embodiments, at least one separate controller 150 may be used for one or all of the subsystems. The controller 150 receives feedback from the subsystems, including the temperature sensor 123 and the pressure sensor 135, and may control operation of any of the elements of all three subsystems.

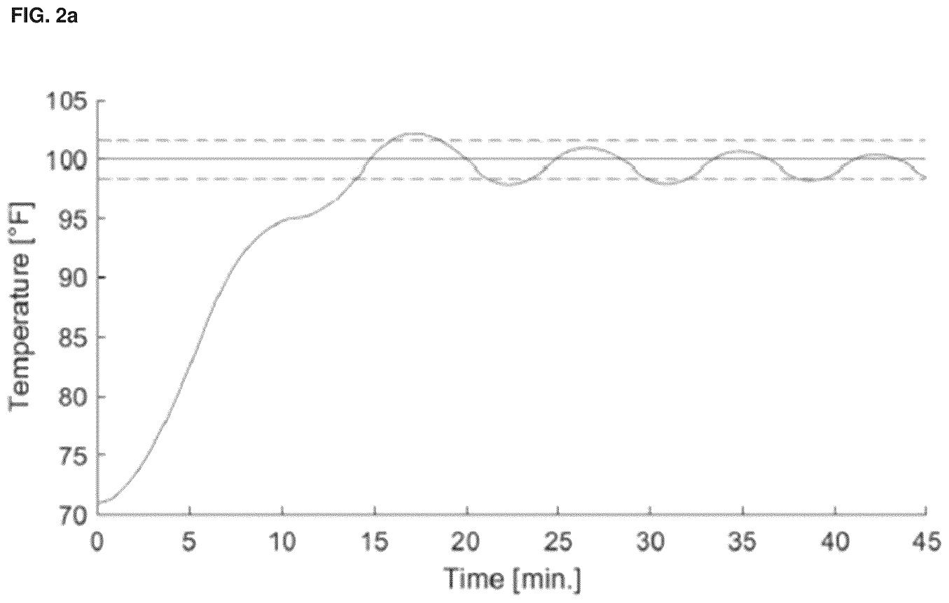

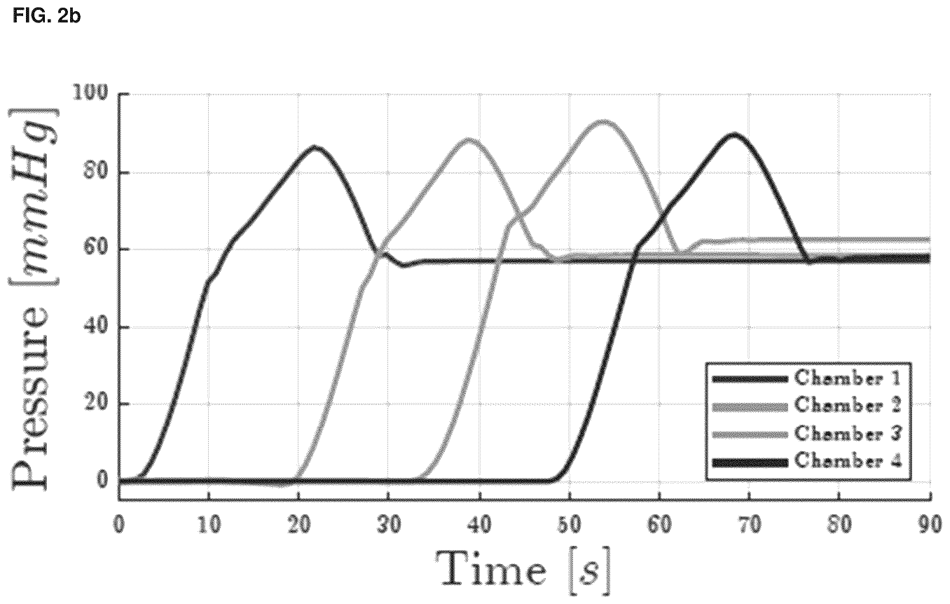

As shown in the graph of FIG. 2a, the controller 150 controls the heating temperature to a predetermined heating maximum, plus or minus a fluctuation value, to apply minimal heating to the periphery and to avoid any adverse patient effects such as tissue burns, heat induced limb ulceration, or any other complications that may arise from excessive, prolonged heat. In an embodiment, the heating elements 122 are controlled with pulse width modulation of power supplied to the heating elements 122 from the power source 160 via the controller 150. In an embodiment, the heating elements 122 are controlled at 100 Hz. In an embodiment, the heating elements 122 are controlled via a solid-state relay switch in the controller 150. In an embodiment, the heating temperature is controlled to a predetermined heating maximum of 100 degrees F. with a fluctuation value of 1 degree F. In certain embodiments, a patient or other user may use the user interface 151 to reduce the heating temperature from the predetermined heating maximum, if the predetermined heating maximum causes discomfort. In certain embodiments, a non-patient user may use the user interface 151 to set the predetermined heating maximum and/or the emergency maximum.

As shown in the graph of FIG. 2b, the controller 150 inflates the pressure chambers 131 cyclically, providing a rhythmic compression to the limb, beginning with the pressure chamber 131 located at the distalmost portion of the limb. Each pressure chamber 131 is inflated sequentially by the pressure source 133 as actuated by the controller 150, beginning with the distalmost pressure chamber 131. Upon inflation, the pressure chambers 131 exert a momentary high pressure on the limb, followed by a steady elevated pressure on the limb. In an embodiment, control of pressure on the limb is ensured by the pressure valves 134 as actuated by the controller 150. In an embodiment, momentary high pressure is approximately 90 mmHg. In an embodiment, steady elevated pressure is approximately 60 mmHg.

The subsequent pressure chamber 131 inflates once the preceding pressure chamber reaches steady elevated pressure. The pressure chambers 131 remain at the steady elevated pressure until all pressure chambers have been inflated and reach steady elevated pressure for a given period of time. The pressure chambers 131 then deflate to exert negligible pressure on the limb, and the cycle of inflation begins again. In certain embodiments, a patient or other user may use the user interface 151 to reduce the momentary high pressure and/or steady elevated pressure, if the momentary high pressure or steady elevated pressure causes discomfort.

In an embodiment, the controller 150 controls each vibrational actuator 141 with pulse width modulation of power supplied to the vibrational actuator 141 from the power source 160. In certain embodiments, a patient or other user may use the user interface 151 to reduce the oscillation frequency and/or amplitude, if the vibrotactile sensation is too intense for comfort.

A user interface 151 operably connected to the controller 150 allows the patient or another user to set the levels and intensity of stimulation from the heating subsystem 120, pressure subsystem 130, and vibration subsystem 140. The user interface 151 may include a graphical user interface, a desktop, a speaker, a mouse, a keyboard, a voice input device, a touch input device for receiving a gesture from a user, a motion input device for detecting non-touch gestures and other motions by a user, and other comparable input/output devices and associated processing elements capable of receiving input and/or producing output.

Certain embodiments may incorporate multiple user interfaces 151, which may have differing permission levels for control of and/or feedback from the heating subsystem 120, pressure subsystem 130, and/or vibration subsystem 140. By way of non-limiting example, a clinical user interface 151 may be able to set a heating maximum for the heating subsystem 120 and observe readings from the temperature sensor 123, while a patient user interface 151 is only able to reduce the heating maximum for the heating subsystem 120 and only receives an alert if the temperature of a heating element 122 exceeds the emergency maximum.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In the foregoing description, certain terms have been used for brevity, clearness, and understanding. No unnecessary limitations are to be inferred therefrom beyond the requirement of the prior art because such terms are used for descriptive purposes and are intended to be broadly construed. The different configurations, systems, and/or method steps described herein may be used alone or in combination with other configurations, systems, and/or method steps. It is to be expected that various equivalents, alternatives, and modifications are possible within the scope of the foregoing description.

What is claimed is:

1. A system for treatment of peripheral neuropathy, comprising:
a heating subsystem comprising at least one heating element;
a pressure subsystem comprising a plurality of pressure chambers;
a vibration subsystem comprising a plurality of vibrational actuators;
a limb sleeves containing the heating subsystem, the pressure subsystem, and the vibration subsystem;
at least one controller operably connected to the heating subsystem, the pressure subsystem, and the vibration subsystem; and
at least one power source.

2. The system of claim 1, wherein the at least one heating element is located within at least one heating pad.

3. The system of claim 1, wherein the at least one heating element is an electrically resistive heating element receiving power from the power source.

4. The system of claim 1, wherein the at least one heating element is a channel for the passage and circulation of a heated fluid.

5. The system of claim 1, wherein the heating subsystem further comprises at least one temperature sensor located proximally to a heating element.

6. The system of claim 5, wherein the heating subsystem further comprises an electrical or physical switch or interrupt configured as a temperature safety to turn off all power to the heating subsystem if a temperature of the at least one heating element rises above a preselected emergency maximum.

7. The system of claim 1, wherein the controller is configured to control the at least one heating element to a heating temperature of a predetermined heating maximum, plus or minus a fluctuation value.

8. The system of claim 7, wherein the at least one heating element is controlled with pulse width modulation.

9. The system of claim 1, wherein the plurality of pressure chambers substantially encircle and sequentially extend along the limb.

10. The system of claim 1, wherein the plurality of pressure chambers are hollow bladders receiving an inflation fluid.

11. The system of claim 1, wherein the controller is configured to inflate the plurality of pressure chambers to exert a momentary high pressure on the limb, followed by a steady elevated pressure on the limb.

12. The system of claim 11, wherein the controller is configured to inflate a subsequent pressure chamber once a preceding pressure chamber reaches steady elevated pressure.

13. The system of claim 11, wherein the controller is configured to retain the plurality of pressure chambers at the steady elevated pressure until all of the plurality of pressure chambers have been inflated and reach steady elevated pressure for a given period of time.

14. The system of claim 1, wherein the plurality of vibrational actuators are attached to at least one motor strap extending around the limb.

15. The system of claim 1, wherein the plurality of vibrational actuators are placed on a single motor strap on a patient's foot, spaced such that one of the plurality of vibrational actuators is placed beneath each metatarsal head of the patient's foot.

16. The system of claim 1, wherein the plurality of vibrational actuators are placed on a single motor strap on a patient's foot, spaced such that one of the plurality of vibrational actuators is placed beneath each distal phalanx of the patient's foot.

17. The system of claim 1, wherein each of the plurality of vibrational actuators is placed on a single motor strap on a patient's calf, spaced along a longitudinal axis of the patient's calf such that the plurality of vibrational actuators extend in a line along the patient's sural nerve.

18. The system of claim 1, wherein each of the plurality of vibrational actuators is connected to the at least one power source by a vibrational actuator fuse.

19. The system of claim 1, wherein the controller is configured to control each of the plurality of vibrational actuators with pulse width modulation.

20. The system of claim 1, further comprising a user interface operably coupled to the at least one controller.
